(19) United States
(12) Patent Application Publication
(10) Pub. No.: US 2023/0201171 A1
(43) Pub. Date: Jun. 29, 2023

(54) COMPOSITIONS AND METHODS FOR PREVENTING POST-ERCP PANCREATITIS

(71) Applicant: THE BOARD OF TRUSTEES OF THE LELAND STANFORD JUNIOR UNIVERSITY, Stanford, CA (US)

(72) Inventors: Sohail Z. Husain, Mountain View, CA (US); Monique T. Barakat, Cupertino, CA (US)

(73) Assignee: THE BOARD OF TRUSTEES OF THE LELAND STANFORD JUNIOR UNIVERSITY, Stanford, CA (US)

(21) Appl. No.: 17/925,985

(22) PCT Filed: Mar. 19, 2021
(86) PCT No.: PCT/US2021/023144

§ 371 Date: Nov. 17, 2022

Related U.S. Application Data
(60) Provisional application No. 63/027,541, filed on May 20, 2020.

(51) Int. Cl. A61K 31/436; A61K 31/405; A61K 9/00; A61K 31/18; A61K 31/60

(52) U.S. Cl. CPC A61K 31/436; A61K 9/0031; A61K 31/18; A61K 31/60; A61K 31/405

(57) ABSTRACT

Provided herein are compositions comprising a calcineurin inhibitor and a nonsteroidal anti-inflammatory drug (NSAID) and related methods of administering such compositions for preventing post-ERCP pancreatitis (PEP).

### CROSS-REFERENCE TO RELATED APPLICATION

This application claims priority to U.S. Provisional Application No. 63/027,541, filed May 20, 2020, the disclosure of which is hereby incorporated by reference in its entirety for all purposes.

### STATEMENT AS TO RIGHTS TO INVENTIONS MADE UNDER FEDERALLY SPONSORED RESEARCH AND DEVELOPMENT

This invention was made with Government support under contract DK093491 awarded by the National Institutes of Health. The Government has certain rights in the invention.

### BACKGROUND

An endoscopic retrograde cholangiopancreatography (ERCP) is a common and life-saving gastrointestinal (GI) procedure that is performed in almost half a million Americans each year. ERCPs are performed by injecting radiocontrast (RC) dye into the biliary or pancreatic duct (PD) through an endoscope. The procedure is essential for several GI emergencies, including the removal of impacted gallstones from the common bile duct and for direct radiographic visualization of the bilio-pancreatic anatomy.

However, post-ERCP pancreatitis (PEP) is a complication that currently occurs at a rate of 3-15% in patients undergoing ERCP. PEP is a life-threatening inflammatory disorder of the pancreas that is excruciatingly painful for patients, and it is a costly, iatrogenic complication that lowers healthcare quality metrics. Although recent PD stenting, anti-inflammatory prophylaxis, and fluid administration have reduced the rate of PEP down to the lower end of the range in some centers. PEP is still the most common adverse effect of ERCP, and the efficacy and practical use of current preventative modalities are still debated.

As such, there is a need for improved therapies for the prevention of PEP that specifically target the underlying mechanisms for PEP. The present disclosure addresses this need and provides associated and other advantages.

### BRIEF SUMMARY

In some aspects, provided herein are compositions comprising a calcineurin inhibitor and a nonsteroidal anti-inflammatory drug (NSAID).

In some embodiments, the calcineurin inhibitor is selected from the group consisting of tacrolimus, ciclosporin, voclosporin, pimecrolimus, a prodrug thereof, an analog thereof, and a combination thereof.

In some embodiments, the NSAID is selected from the group consisting of an acetic acid derivative, a propionic acid derivative, an enolic acid derivative, an anthranilic acid derivative, a salicylate, a selective COX-2 inhibitor, a sulfonanilide, a prodrug thereof, an analog thereof, and a combination thereof.

Non-limiting examples of acetic acid derivatives include indomethacin, ketorolac, sulindac, tolmetin, etodolac, diclofenac, aceclofenac, bromfenac, nabumetone, and a combination thereof.

Non-limiting examples of propionic acid derivatives include ibuprofen, naproxen, ketoprofen, dexibuprofen, fenoprofen, dexketoprofen, flurbiprofen, oxaprozin, loxoprofen, and a combination thereof.

Non-limiting examples of enolic acid derivatives include meloxicam piroxicam, tenoxicam, droxicam, lornoxicam, isoxicam, phenylbutazone, and a combination thereof.

Non-limiting examples of anthranilic acid derivatives include mefenamic acid, meclofenamic acid, flufenamic acid, tolfenamic acid, and a combination thereof.

Non-limiting examples of salicylates include aspirin, diflunisal, salicylic acid, salsalate, and a combination thereof.

Non-limiting examples of selective COX-2 inhibitors include celecoxib, rofecoxib, valdecoxib, parecoxib, lumiracoxib, etoricoxib, firocoxib and a combination thereof.

A non-limiting example of a sulfonanilide is nimesulide.

In certain embodiments, the calcineurin inhibitor is tacrolimus and/or the NSAID is indomethacin. In certain other embodiments, the calcineurin inhibitor is ciclosporin and/or the NSAID is indomethacin. In particular embodiments, the calcineurin inhibitor is tacrolimus and the NSAID is indomethacin.

In some embodiments, the composition comprises effective amounts of the calcineurin inhibitor and the NSAID to prevent PEP in a subject.

In some embodiments, the composition further comprises a pharmaceutically acceptable carrier. In some embodiments, the pharmaceutically acceptable carrier is suitable for rectal administration. In certain embodiments, the composition is a suppository.

In other aspects, provided herein are methods of administering the compositions described herein to a subject for preventing post-ERCP pancreatitis (PEP).

In some embodiments, the subject is about to undergo ERCP. In certain embodiments, the composition is administered before ERCP is performed on the subject. In other embodiments, the administering is by rectal administration. In particular embodiments, the subject is a human.

In further aspects, provided herein are compositions for preventing PEP in a subject comprising a calcineurin inhibitor and a pharmaceutically acceptable carrier suitable for rectal administration. In related aspects, provided herein are methods for preventing PEP in a subject comprising administering such compositions. In certain embodiments, the subject (e.g., a human) is administered the composition based on clinical context and/or physician judgment.

In some embodiments, the calcineurin inhibitor is selected from the group consisting of tacrolimus, ciclosporin, voclosporin, pimecrolimus, a prodrug thereof, an analog thereof, and a combination thereof. In certain embodiments, the composition is a suppository.

In some embodiments, the subject has a contraindication for NSAID (e.g.. indomethacin) administration. Non-limiting examples of contraindications include pregnancy, a coagulopathy, an anti-coagulation requirement, an NSAID sensitivity or allergy, and a combination thereof.

### BRIEF DESCRIPTION OF THE DRAWINGS

None.

### DETAILED DESCRIPTION

### I. Introduction

We have discovered that an important early signaling pathway for post-ERCP pancreatitis (PEP) is the activation of the calcium-activated phosphatase calcineurin. In novel experimental models, we demonstrated that PEP is dependent on calcineurin. We also found that PEP is prevented through genetic deletion of calcineurin specifically within the pancreas or, importantly, by pharmacological administration of calcineurin inhibitors to the vicinity of the pancreas.

Rectal administration provides a route for enriching delivery of prophylactic agents to the pancreas since there is direct absorption into the portal circulation and therefore flow to the pancreas. A current standard of care for PEP prevention at many institutions is to administer rectal indomethacin, which is thought to reduce distinct aspects of pancreatic inflammation that ensue after the onset of PEP, including cyclo-oxygenase and phospholipase A2 pathways.

The present disclosure relates to compositions comprising a combination of a calcineurin inhibitor (e.g.. tacrolimus, ciclosporin) and a nonsteroidal anti-inflammatory drug (NSAID) (e.g.. indomethacin) as well as use thereof for preventing PEP. Without being bound by the following theory, the compositions are particularly advantageous because they target two broad independent inflammatory pathways: the calcineurin inhibitor intercepts early inflammatory signals involving calcineurin that initiate pancreatic inflammation and the NSAID targets later independent inflammatory signals. The compositions can be formulated with a pharmaceutically acceptable carrier suitable for rectal administration. e.g., as a suppository, to directly target the pancreatic circulation via the portal vein. The compositions can be administered just before the start of an ERCP to any patient undergoing the procedure.

The compositions described herein for PEP prophylaxis provide a number of competitive advantages over current therapies as they are less time-consuming and safer than pancreatic duct stenting (e.g.. risk of complications, repeat procedure) and intravenous (IV) hydration (often contraindicated), twice as effective as rectal indomethacin alone (e.g., >75% vs. 35-40%), ready-to-use and minimally invasive formulations (e.g.. rectal administration), and provide cost-savings due to the reduced hospitalization and morbidity.

### II. Definitions

As used herein, the following terms have the meanings ascribed to them unless specified otherwise.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an agent” or “the agent” includes a plurality of such agents.

The term “about” in relation to a numerical value x means, for example, x±10%.

The term “calcineurin inhibitor” refers to a member of a class of drugs that inhibits the activity of calcineurin, a calcium- and calmodulin-dependent serine/threonine protein phosphatase. Non-limiting examples of calcineurin inhibitors include tacrolimus (FK-506), ciclosporin (ciclosporin A or CsA), voclosporin, pimecrolimus, prodrugs thereof, analogs thereof, derivatives thereof, and combinations thereof.

The term “nonsteroidal anti-inflammatory drug” or “NSAID” refers to a member of a class of drugs that inhibits the activity of one or more cyclooxygenase enzymes (e.g., COX-1 and/or COX-2), which are involved in the synthesis of key biological mediators of inflammation, e.g.. prostaglandins. NSAIDs can be classified based on their chemical structure or mechanism of action. Non-limiting examples of NSAIDs classified by chemical structure include acetic acid derivatives, propionic acid derivatives, enolic acid derivatives, anthranilic acid derivatives, salicylates, sulfonanilides, prodrugs thereof, analogs thereof, and combinations thereof. Non-limiting examples of NSAIDs classified by mechanism of action include selective COX-2 inhibitors, prodrugs thereof, analogs thereof, derivatives thereof, and combinations thereof.

The term “endoscopic retrograde cholangiopancreatography” or “ERCP” refers to a procedure that combines the use of endoscopy and fluoroscopy to diagnose and treat certain problems of the biliary or pancreatic ductal systems. ERCP is used primarily to diagnose and treat conditions of the bile ducts and main pancreatic duct, including gallstones, inflammatory strictures (scars), leaks (from trauma and surgery), and cancer.

The term “post-ERCP pancreatitis” or “PEP” refers to the presence of pancreatitis (i.e., new pancreatic-type abdominal pain accompanied by at least a threefold increase in serum amylase levels) that has developed de novo 24 hours after ERCP. PEP is the most common complication of ERCP and can lead to significant morbidity as well as occasional mortality.

The term “preventing” or “prevent” refers to any one of the following: ameliorating one or more symptoms of PEP; precluding the manifestation of such symptoms before they occur; slowing down or completely stopping the progression of PEP; delaying the onset or severity of PEP; or any combination thereof.

The term “administer,” “administering,” or “administration” refers to the methods that may be used to enable delivery of the compositions described herein to a desired site of biological action. These methods include, but are not limited to, parenteral administration (e.g., intravenous, subcutaneous, intraperitoneal, intramuscular, intra-arterial, intravascular, intracardiac, intrathecal, intranasal, intradermal, intravitreal, and the like), transmucosal administration, oral administration, rectal administration (e.g.. as a suppository), and topical administration. One skilled in the art will know of additional methods for administering an effective amount of the compositions described herein for preventing PEP or relieving one or more symptoms associated with PEP.

The term “effective amount” or “effective dose” refers to an amount of a composition or an agent (e.g.. calcineurin inhibitor or NSAID) described herein that is sufficient to bring about a beneficial or desired clinical effect, e.g.. prevention of PEP in a subject. An effective amount or dose may be based on factors individual to each patient, including, but not limited to, the patient’s age, size, type or extent of gastrointestinal (GI) condition or disease, and route of administration of the composition. Effective amounts of the agents can be estimated initially from cell culture and animal models. For example, IC₅₀ values determined in cell culture methods can serve as a starting point in animal models, while IC₅₀ values determined in animal models can be used to find an effective dose in humans.

The term “pharmaceutically acceptable carrier” refers to a carrier or a diluent that does not cause significant irritation to a subject and does not abrogate the biological activity and properties of the administered agent or agents.

The terms “subject.” “individual,” and “patient” are used interchangeably herein to refer to a vertebrate, preferably a mammal, more preferably a human. Mammals include, but are not limited to, murines, rats, simians, humans, farm animals, sport animals, and pets.

### III. Pharmaceutical Compositions

The compositions described herein comprise a combination of agents (e.g.. calcineurin inhibitor and NSAID) and may further comprise a pharmaceutically acceptable carrier. In certain aspects, pharmaceutically acceptable carriers are determined in part by the particular composition being administered, as well as by the particular method used to administer the composition. Accordingly, there is a wide variety of suitable formulations of pharmaceutical compositions of the present invention (see. e.g., REMINGTON’S PHARMACEUTICAL SCIENCES, 18TH ED., Mack Publishing Co., Easton, PA (1990)).

As used herein, a pharmaceutically acceptable carrier comprises any of standard pharmaceutically accepted carriers known to those of ordinary skill in the art in formulating pharmaceutical compositions. Thus, the agents, by themselves, such as being present as pharmaceutically acceptable salts, or as conjugates, may be prepared as formulations in pharmaceutically acceptable diluents: for example, saline, phosphate buffer saline (PBS), aqueous ethanol, or solutions of glucose, mannitol, dextran, propylene glycol, oils (e.g., vegetable oils, animal oils, synthetic oils, etc.), microcrystalline cellulose, carboxymethyl cellulose, hydroxylpropyl methyl cellulose, magnesium stearate, calcium phosphate, gelatin, polysorbate 80 or the like, or as solid formulations in appropriate excipients.

In certain embodiments, the pharmaceutical compositions comprise one or more buffers (e.g., neutral buffered saline or phosphate buffered saline), carbohydrates (e.g., glucose, mannose, sucrose or dextrans), mannitol, proteins, polypeptides or amino acids such as glycine, antioxidants (e.g., ascorbic acid, sodium metabisulfite, butylated hydroxytoluene, butylated hydroxyanisole, etc.), bacteriostats, chelating agents such as EDTA or glutathione, solutes that render the formulation isotonic, hypotonic or weakly hypertonic with the blood of a recipient, suspending agents, thickening agents, preservatives, flavoring agents, sweetening agents, and coloring compounds as appropriate.

The pharmaceutical compositions are administered in a manner compatible with the dosage formulation, and in such amount as will be effective. The quantity to be administered depends on a variety of factors including, e.g., the age, body weight, physical activity, and diet of the individual, and the type, extent, or severity of gastrointestinal (GI) condition or disease. In certain embodiments, the size of the dose may also be determined by the existence, nature, and extent of any adverse side effects that accompany the administration of the agents in a particular individual.

It will be understood, however, that the specific dose level and frequency of dosage for any particular patient may be varied and will depend upon a variety of factors including the activity of the specific agents employed, the metabolic stability and length of action of those agents, the age, body weight, hereditary characteristics, general health, sex, diet, mode and time of administration, rate of excretion, drug combination, the severity of the particular condition, and the host undergoing the ERCP procedure.

In certain embodiments, the dose of the agents may take the form of solid, semi-solid, lyophilized powder, or liquid dosage forms, such as, for example, tablets, pills, pellets, capsules, powders, solutions, suspensions, emulsions, suppositories, retention enemas, creams, ointments, lotions, gels, aerosols, foams, or the like, preferably in unit dosage forms suitable for simple administration of precise dosages.

As used herein, the term “unit dosage form” refers to physically discrete units suitable as unitary dosages for humans and other mammals, each unit containing a predetermined quantity of the agents calculated to produce the desired onset, tolerability, and/or effects, in association with a suitable pharmaceutical excipient. In addition, more concentrated dosage forms may be prepared, from which the more dilute unit dosage forms may then be produced. The more concentrated dosage forms thus will contain substantially more than, e.g., at least 1, 2, 3, 4, 5, 6, 7, 8, 9, 10, or more times the amount of the agents.

Methods for preparing such dosage forms are known to those skilled in the art (see, e.g.. REMINGTON’S PHARMACEUTICAL SCIENCES, supra). The dosage forms typically include a conventional pharmaceutical carrier or excipient and may additionally include other medicinal agents, carriers, adjuvants, diluents, tissue permeation enhancers, solubilizers, and the like. Appropriate excipients can be tailored to the particular dosage form and route of administration by methods well known in the art (see, e.g.. REMINGTON’S PHARMACEUTICAL SCIENCES, supra).

Examples of suitable excipients include, but are not limited to, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, saline, syrup, methylcellulose, ethylcellulose, hydroxypropylmethylcellulose, and polyacrylic acids such as Carbopols, e.g., Carbopol 941, Carbopol 980, Carbopol 981, etc. The dosage forms can additionally include lubricating agents such as talc, magnesium stearate, and mineral oil; wetting agents; emulsifying agents; suspending agents; preserving agents such as methyl-, ethyl-, and propyl-hydroxy-benzoates (i.e., the parabens); pH adjusting agents such as inorganic and organic acids and bases; sweetening agents; and flavoring agents. The dosage forms may also comprise biodegradable polymer beads, dextran, and cyclodextrin inclusion complexes.

Rectal preparations can be liquid, semi-solid, or solid preparations. For rectal administration, the effective dose can be in the form of suppositories, rectal capsules, rectal solutions, emulsions, or suspensions (e.g.. retention enemas), powders or tablets for rectal solutions or suspensions, and semi-solid rectal preparations such as creams, ointments, gels, and foams. Rectal administration may also be performed using a specialized catheter designed for delivery of medication to the rectum. Any excipient that does not adversely affect the stability of the preparation nor the bioavailability of the agents at the site of action can be used.

Suppositories can contain the agents dispersed or dissolved in a suitable base that may be soluble or dispersible in water or may melt at body temperature. When prepared by molding, suppository bases such as macrogols, gelatinous mixtures consisting of, for example, gelatin, water and glycerol, hydrogenated vegetable oils, hard fat or cocoa butter are usually employed. Excipients such as diluents, adsorbents, lubricants, antimicrobial preservatives, and coloring matter may be added.

Rectal capsules are solid, single-dose preparations generally similar to soft capsules except that they may have a lubricating coating. The contents of rectal capsules are usually solutions or suspensions of the agents in non-aqueous liquids, e.g., vegetable oil, or in semi-solid mixtures of suitable excipients.

Rectal solutions, emulsions, and suspensions (also called enemas) are liquid preparations intended for rectal application and can contain the agents dissolved or dispersed in water, glycerol, macrogols, vegetable oil, or mixtures thereof. They may contain excipients, for example, to adjust the viscosity of the preparation, to adjust or stabilize pH, to increase the solubility of the agents, and/or to stabilize the preparation. Rectal solutions, emulsions, and suspensions are typically supplied in single-dose containers containing a volume in the range of about 2.5 mL to about 2000 mL. The container can be adapted to deliver the preparation to the rectum or is accompanied by a suitable applicator.

Powders and tablets intended for the preparation of rectal solutions or suspensions are single-dose preparations that are dissolved or dispersed in water or other suitable solvents at the time of administration. They may contain excipients to facilitate dissolution or dispersion or to prevent aggregation of the particles.

Semi-solid rectal preparations include ointments, creams, gels, and foams intended for local treatment in the rectum. They are usually supplied as single-dose preparations in containers adapted to deliver the preparation to the rectum or are accompanied by a suitable applicator.

For oral administration, the effective dose can be in the form of tablets, capsules, emulsions, suspensions, solutions, syrups, sprays, lozenges, powders, and sustained-release formulations. Suitable excipients for oral administration include pharmaceutical grades of mannitol, lactose, starch, magnesium stearate, sodium saccharine, talcum, cellulose, glucose, gelatin, sucrose, magnesium carbonate, and the like.

The effective dose can also be provided in a lyophilized form. Such dosage forms may include a buffer, e.g.. bicarbonate, for reconstitution prior to administration, or the buffer may be included in the lyophilized dosage form for reconstitution with, e.g., water. The lyophilized dosage form may further comprise a suitable vasoconstrictor, e.g.. epinephrine. The lyophilized dosage form can be provided in a syringe, optionally packaged in combination with the buffer for reconstitution, such that the reconstituted dosage form can be immediately administered to an individual.

In some embodiments, a pharmaceutical composition of the present invention comprises a pharmaceutically acceptable carrier that comprises an aqueous base. In other embodiments, the pharmaceutically acceptable carrier comprises a low viscosity compound. In some instances, the low viscosity compound comprises gelatin. In other instances, the low viscosity compound comprises a hydrogel.

### IV. Methods of Administration

The compositions described herein can be administered to a subject for preventing post-ERCP pancreatitis (PEP). The compositions can be administered via enteral administration (e.g., oral, buccal, sublingual, or rectal), parenteral administration (e.g.. intravenous, intramuscular, intra-arterial, intradermal, subcutaneous, intraperitoneal, intraventricular, intraosseous, or intracranial), or transmucosal administration (e.g.. nasal, vaginal, or transdermal). Other modes of delivery include, but are not limited to, the use of liposomal formulations, intravenous infusion, transdermal patches, etc. In particular embodiments, the compositions are administered rectally.

In some embodiments, the composition is administered once to a subject, for example, before ERCP is performed on the subject. The composition can be administered at least about 5, 10, 15, 20, 25, 30, 40, 50, 60, 70, 80, 90, 100, 110, or 120 minutes before ERCP is performed on the subject. In particular embodiments, the composition is administered immediately following the induction of anesthesia, as part of the preparation and positioning of the subject for ERCP.

In some embodiments, the composition may be administered at dosage levels sufficient to deliver from about 0.0001 mg/kg to about 100 mg/kg, from about 0.001 mg/kg to about 50 mg/kg, from about 0.01 mg/kg to about 50 mg/kg, from about 0.05 mg/kg to about 0.5 mg/kg, from about 0.1 mg/kg to about 50 mg/kg, from about 0.01 mg/kg to about 10 mg/kg, from about 0.1 mg/kg to about 10 mg/kg, or from about 1 mg/kg to about 10 mg/kg, of the subject’s body weight to obtain the desired prophylactic effect.

In some embodiments, the composition contains a dose of the NSAID (e.g., indomethacin) and/or a dose of the calcineurin inhibitor (e.g.. tacrolimus) each independently comprising about 0.1, 0.5, 1, 5, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, or more mg of the agent. In particular embodiments, the composition contains 100 mg of an NSAID such as indomethacin.

In some embodiments, the composition contains a dose of the NSAID (e.g.. indomethacin) and/or a dose of the calcineurin inhibitor (e.g., tacrolimus) each independently comprising an equivalent dose corresponding to the dose of the agent present in a reference dosage form. In certain embodiments, the reference dosage form contains about 0.1, 0.5, 1, 5, 10, 20, 30, 40, 50, 60, 70, 80, 90, 100, 110, 120, 130, 140, 150, 160, 170, 180, 190, 200, 250, 300, 350, 400, 450, 500, 550, 600, 650, 700, 750, 800, 850, 900, 950, 1000, or more mg of the agent. In particular embodiments, the composition contains a calcineurin inhibitor such as tacrolimus at an equivalent dose corresponding to 15 mg of tacrolimus oral powder.

In some embodiments, rectal administration of the composition to the subject prior to ERCP results in about a 50%, 55%, 60%, 65%, 70%, 75%, 80%, 85%, 90%, 95%, or greater reduction in the rate of PEP relative to the current standard of care (e.g., rectal indomethacin alone). In particular embodiments, rectal administration of the composition to the subject prior to ERCP results in about a 75% or greater reduction in the rate of PEP relative to rectal indomethacin alone.

### V. Examples

The present invention will be described in greater detail by way of specific examples. The following examples are offered for illustrative purposes only, and are not intended to limit the invention in any manner.

### Example 1. Formulation of Rectal Composition Comprising Tacrolimus and Indomethacin

To optimize delivery of a composition comprising tacrolimus and indomethacin via the rectum, components are selected which result in rapid and predictable delivery of these agents. The rectum is relatively constant and static in comparison to other parts of the gastrointestinal tract, which represents an advantage of this delivery modality. The rectum has an average fluid volume of 1-3 mL and a neutral pH of 7-8, with minimal buffering capacity, and the vehicle for delivery of tacrolimus and indomethacin takes this into account. An exemplary composition includes 100 mg indomethacin and an equivalent dose corresponding to 15 mg of tacrolimus oral powder.

### Example 2. Prevention of PEP With Rectal Composition Comprising Tacrolimus And Indomethacin

The tacrolimus and indomethacin rectal formulation is administered to patients immediately following induction of anesthesia, as part of standard patient preparation and positioning for ERCP. This administration time frame integrates seamlessly within the process of patient preparation prior to ERCP and also results in optimal, predictable, therapeutic local and systemic levels of both agents during the ERCP-related instrumentation and contrast injection which heightens the risk for post-ERCP pancreatitis (PEP). Administration of the formulation prior to ERCP results in about a 75% or greater reduction in the rate of PEP relative to the current standard of care.

### VI. Exemplary Embodiments

Exemplary embodiments provided in accordance with the presently disclosed subject matter include, but are not limited to, the claims and the following embodiments:
1. A composition comprising a calcineurin inhibitor and a nonsteroidal anti-inflammatory drug (NSAID).
2. The composition of embodiment 1, wherein the calcineurin inhibitor is selected from the group consisting of tacrolimus, ciclosporin, voclosporin, pimecrolimus, a prodrug thereof, an analog thereof, and a combination thereof.
3. The composition of embodiment 1 or 2, wherein the NSAID is selected from the group consisting of an acetic acid derivative, a propionic acid derivative, an enolic acid derivative, an anthranilic acid derivative, a salicylate, a selective COX-2 inhibitor, a sulfonanilide, a prodrug thereof, an analog thereof, and a combination thereof.
4. The composition of embodiment 3, wherein the acetic acid derivative is selected from the group consisting of indomethacin, ketorolac, sulindac, tolmetin, etodolac, diclofenac, aceclofenac, bromfenac, nabumetone, and a combination thereof.
5. The composition of embodiment 3, wherein the propionic acid derivative is selected from the group consisting of ibuprofen, naproxen, ketoprofen, dexibuprofen, fenoprofen, dexketoprofen, flurbiprofen, oxaprozin, loxoprofen, and a combination thereof.
6. The composition of embodiment 3, wherein the enolic acid derivative is selected from the group consisting of meloxicam piroxicam, tenoxicam, droxicam, lornoxicam, isoxicam, phenylbutazone, and a combination thereof.
7. The composition of embodiment 3, wherein the anthranilic acid derivative is selected from the group consisting of mefenamic acid, meclofenamic acid, flufenamic acid, tolfenamic acid, and a combination thereof.
8. The composition of embodiment 3. wherein the salicylate is selected from the group consisting of aspirin, diflunisal, salicylic acid, salsalate, and a combination thereof.
9. The composition of embodiment 3. wherein the selective COX-2 inhibitor is selected from the group consisting of celecoxib, rofecoxib, valdecoxib, parecoxib, lumiracoxib, etoricoxib, firocoxib and a combination thereof.
10. The composition of embodiment 3, wherein the sulfonanilide is nimesulide.
11. The composition of embodiment 1, wherein the calcineurin inhibitor is tacrolimus and/or the NSAID is indomethacin.
12. The composition of any one of embodiments 1 to 11, wherein the composition comprises effective amounts of the calcineurin inhibitor and the NSAID to prevent post-endoscopic retrograde cholangiopancreatography (ERCP) pancreatitis (PEP) in a subject.
13. The composition of any one of embodiments 1 to 12, further comprising a pharmaceutically acceptable carrier.
14. The composition of embodiment 13, wherein the pharmaceutically acceptable carrier is suitable for rectal administration.
15. The composition of any one of embodiments 1 to 14, wherein the composition is a suppository.
16. A method for preventing PEP in a subject, the method comprising administering the composition of any one of embodiments 1 to 15 to the subject.
17. The method of embodiment 16, wherein the subject is about to undergo ERCP.
18. The method of embodiment 16 or 17, wherein the composition is administered before ERCP is performed on the subject.
19. The method of any one of embodiments 16 to 18, wherein the administering is by rectal administration.
20. A composition for preventing PEP in a subject, the composition comprising a calcineurin inhibitor and a pharmaceutically acceptable carrier suitable for rectal administration.
21. The composition of embodiment 20. wherein the calcineurin inhibitor is selected from the group consisting of tacrolimus, ciclosporin, voclosporin, pimecrolimus, a prodrug thereof, an analog thereof, and a combination thereof.
22. The composition of embodiment 20 or 21, wherein the composition is a suppository.
23. The composition of any one of embodiments 20 to 22, wherein the subject has a contraindication for indomethacin administration.
24. The composition of embodiment 23. wherein the contraindication for indomethacin administration is selected from the group consisting of pregnancy, a coagulopathy, an anti-coagulation requirement, an NSAID sensitivity or allergy, and a combination thereof.
25. A method for preventing PEP in a subject, the method comprising administering the composition of any one of embodiments 20 to 24 to the subject.
26. The method of embodiment 25, wherein the subject is a human.
27. The method of embodiment 26, wherein the subject is administered the composition based on clinical context and/or physician judgment.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

What is claimed is:

1. A composition comprising a calcineurin inhibitor and a nonsteroidal anti-inflammatory drug (NSAID).

2. The composition of claim 1, wherein the calcineurin inhibitor is selected from the group consisting of tacrolimus, ciclosporin, voclosporin, pimecrolimus, a prodrug thereof, an analog thereof, and a combination thereof.

3. The composition of claim 1, wherein the NSAID is selected from the group consisting of an acetic acid derivative, a propionic acid derivative, an enolic acid derivative, an anthranilic acid derivative, a salicylate, a selective COX-2 inhibitor, a sulfonanilide, a prodrug thereof, an analog thereof, and a combination thereof.

4. The composition of claim 3, wherein the acetic acid derivative is selected from the group consisting of indomethacin, ketorolac, sulindac, tolmetin, etodolac, diclofenac, aceclofenac, bromfenac, nabumetone, and a combination thereof.

5. The composition of claim 3, wherein the propionic acid derivative is selected from the group consisting of ibuprofen, naproxen, ketoprofen, dexibuprofen, fenoprofen, dexketoprofen, flurbiprofen, oxaprozin, loxoprofen, and a combination thereof.

6. The composition of claim 3, wherein the enolic acid derivative is selected from the group consisting of meloxicam piroxicam, tenoxicam, droxicam, lomoxicam, isoxicam, phenylbutazone, and a combination thereof.

7. The composition of claim 3, wherein the anthranilic acid derivative is selected from the group consisting of mefenamic acid, meclofenamic acid, flufenamic acid, tolfenamic acid, and a combination thereof.

8. The composition of claim 3, wherein the salicylate is selected from the group consisting of aspirin, diflunisal, salicylic acid, salsalate, and a combination thereof.

9. The composition of claim 3, wherein the selective COX-2 inhibitor is selected from the group consisting of celecoxib, rofecoxib, valdecoxib, parecoxib, lumiracoxib, etoricoxib, firocoxib and a combination thereof.

10. The composition of claim 3, wherein the sulfonanilide is nimesulide.

11. The composition of claim 1, wherein the calcineurin inhibitor is tacrolimus and/or the NSAID is indomethacin.

12. The composition of claim 1, wherein the composition comprises effective amounts of the calcineurin inhibitor and the NSAID to prevent post-endoscopic retrograde cholangiopancreatography (ERCP) pancreatitis (PEP) in a subject.

13. The composition of claim 1, further comprising a pharmaceutically acceptable carrier.

14. The composition of claim 13, wherein the pharmaceutically acceptable carrier is suitable for rectal administration.

15. The composition of claim 1, wherein the composition is a suppository.

16. A method for preventing PEP in a subject, the method comprising administering the composition of claim 1 to the subject.

17. The method of claim 16, wherein the subject is about to undergo ERCP.

18. The method of claim 16, wherein the composition is administered before ERCP is performed on the subject.

19. The method of claim 16, wherein the administering is by rectal administration.

20. A composition for preventing PEP in a subject, the composition comprising a calcineurin inhibitor and a pharmaceutically acceptable carrier suitable for rectal administration.

21. The composition of claim 20, wherein the calcineurin inhibitor is selected from the group consisting of tacrolimus, ciclosporin, voclosporin, pimecrolimus, a prodrug thereof, an analog thereof, and a combination thereof.

22. The composition of claim 20, wherein the composition is a suppository.

23. The composition of claim 20, wherein the subject has a contraindication for indomethacin administration.

24. The composition of claim 23, wherein the contraindication for indomethacin administration is selected from the group consisting of pregnancy, a coagulopathy, an anti-coagulation requirement, an NSAID sensitivity or allergy, and a combination thereof.

25. A method for preventing PEP in a subject, the method comprising administering the composition of claim 20 to the subject.

26. The method of claim 25, wherein the subject is a human.

27. The method of claim 26, wherein the subject is administered the composition based on clinical context and/or physician judgment.
